(19) United States
(12) Patent Application Publication
(10) Pub. No.: US 2024/0018568 A1
(43) Pub. Date: Jan. 18, 2024

(54) Microfluidic Analysis Device and Method for the Operation Thereof

(71) Applicant: Robert Bosch GmbH, Stuttgart (DE)

(72) Inventors: Yvonne Beyl, Zaberfeld (DE); Franz Laermer, Weil Der Stadt (DE); Stefanie Wagner, Ludwigsburg (DE); Anke Timm, Renningen (DE); Jochen Hoffmann, Renningen (DE); Martina Budde, Karlsruhe (DE); Daniel Sebastian Podbiel, Rutesheim (DE)

(21) Appl. No.: 18/250,689

(22) PCT Filed: Oct. 27, 2021
(86) PCT No.: PCT/EP2021/079746

§ 371 Date: Apr. 26, 2023

(30) Foreign Application Priority Data
Oct. 27, 2020 (DE) 10 2020 213 472.3

(51) Int. Cl. C12Q 1/6806; C12Q 1/6844; B01L 7/00; B01L 3/00

(52) U.S. Cl. CPC C12Q 1/6806; C12Q 1/6844; B01L 7/525; B01L 3/5027; B01L 2200/0647; B01L 2300/0861

(57) ABSTRACT

The disclosure relates to a microfluidic analysis device and to a method for operating the microfluidic analysis device. The method comprises the following steps: providing a sample containing DNA, performing a PCR pre-amplification of the sample, dividing the sample into at least two reaction compartments, and performing at least one singleplex detection in each of the at least two reaction compartments. The singleplex detection is performed in each case by means of an isothermal amplification system.

The invention relates to a method for operating a microfluidic analysis device. Furthermore, the present invention relates to a microfluidic analysis device configured to be operated by means of the method.

### PRIOR ART

Microfluidic analysis devices, which are also called lab-on-chips (LoCs), enable automated, reliable, fast, compact, and cost-effective processing of samples for medical diagnostics. By combining a plurality of operations for controlled manipulation of fluids, complex molecular diagnostic test procedures can be performed on a cartridge of a microfluidic analysis device.

In the molecular diagnostic analysis of a sample, the sample is to be examined for a plurality of genetic characteristics at high sensitivity. This is referred to as multiplexed detection of various gene targets or nucleic acid sequences. This may be done based on amplification reactions for specifically duplicating predetermined nucleic acid sequences, such as, in particular, the polymerase chain reaction (PCR). A so-called nested PCR is based on a two-step detection of the gene targets. In this case, longer DNA sequences, which may include several DNA targets, are first duplicated in a PCR pre-amplification. Thereafter, dilution, aliquoting and distribution of the reaction mixture with the amplified nucleic acid sequences to a plurality of reaction compartments take place, each with target-specific primers for PCR detection in singleplex format. By combining multiplex pre-amplification and detection of the targets in singleplex format, a high sensitivity on the one hand and a high multiplex degree on the other hand can be achieved.

However, for performing the individual singleplex PCR detection reactions, precisely timed temperature control of the individual reaction compartments is necessary, which requires a good thermal coupling of the reaction compartments to at least one external heating and/or cooling device. However, polymeric materials as used to inexpensively produce cartridges of microfluidic analysis devices typically have only low thermal conductivity. This leads to long heating and cooling times and thus to a high overall time expenditure for such PCR detection.

### DISCLOSURE OF THE INVENTION

In the method for operating a microfluidic analysis device, a sample containing DNA is first provided. This is in particular a patient sample. If the target nucleic acid to be amplified in the method is a ribonucleic acid (RNA), reverse transcription, which transcribes the RNA into DNA, is first performed prior to the provision of the sample. Subsequently, PCR pre-amplification of the sample is performed in order to generate a sufficient amount of DNA for subsequently performing singleplex detections. If necessary, the sample can be diluted before the sample is divided or aliquoted into at least two reaction compartments in the next step. At least one singleplex detection is then performed in each of the reaction compartments. In this respect, the steps of the method correspond to a conventional operation of a microfluidic analysis device using multiplex pre-amplification and several singleplex detections.

However, it has been found that the problem of long heating and cooling times can be overcome in such a method by performing the singleplex detections by means of an isothermal amplification system in each case. In this way, cyclic tempering of the plurality of reaction compartments is no longer required. Thus, it is unproblematic if the material of the microfluidic analysis device surrounding the reaction compartments has only low thermal conductivity and/or is surrounded by a material having only low thermal conductivity. This results a significant advantage over the prior art.

By performing the isothermal detections in singleplex format, a particularly simple design of the isothermal detection reactions is possible. Performing the singleplex detection reactions independently of one another allows for a particularly simple adjustment of the detection panel of an assay by adding or replacing singleplex detection reactions. Furthermore, a particularly simple adjustment of the detection panel is made possible by simply adding or replacing corresponding primer pairs in the PCR pre-amplification, which in particular results from the PCR pre-amplification operating only at low amplification and different amplification factors for individual reaction products therefore not carrying as much weight as in a PCR pre-amplification that is controlled into deep saturation.

For example, suitable isothermal amplification systems that can be used in the method for performing the singleplex detection are selected from the group consisting of SDA (strand displacement amplification), RPA (recombinase polymerase amplification), LAMP (loop-mediated isothermal amplification), RCA (rolling cycle amplification), MDA (multidisplacement amplification), NASBA (nucleic acid sequence-based amplification), HDA (helicase-dependent amplification), NEAR (nicking enzyme amplification reaction), NESA (nicking endonuclease signal amplification), and HCR (hybridization chain reaction).

It is preferred that the reaction compartments each have a volume of less than 1 μl. Particularly preferably, the volume is less than 100 nl, and more particularly preferably, it is in the range between 10 nl and 50 nl. Such a small volume is sufficient for singleplex detection in combination with pre-amplification in order to enable both sensitive and multiplexed detection of a plurality of DNA targets. In particular, a particularly small volume of a sample liquid for analysis can be used in this way. Conversely, in the context of microfluidic processing, a low transfer efficiency in the production of the microfluidic reaction compartments is already sufficient to enable analysis. Furthermore, with a given thermal conductivity of the liquid and of the device, such a small volume can be brought particularly quickly to the temperature required for the isothermal amplification system.

Furthermore, it is preferred that the sample is divided into 5 to 500 reaction compartments, particularly preferably into 10 to 200 reaction compartments. This enables the sample to be examined for a plurality of different gene targets. Due to the small volumes of the reaction compartments, this is furthermore possible without the volume of the total sample having to be so large that its heating or cooling during the PCR pre-amplification would lead to a significant loss of time.

The sample is preferably divided using at least a partially fluorinated hydrocarbon. Such hydrocarbons are chemically inert and are therefore well suited as transport media and/or for a thermally stable seal of the reaction compartments. In addition to fully fluorinated perfluorocarbons, such as bis(nonafluorobutyl)-(trifluoromethyl)amine (Fluorinert FC-40 from 3M), partially fluorinated hydrocarbons that carry at least one unsubstituted alkyl group, such as ethyl perfluoroheptyl ether (Novec 7500 from 3M), in addition to at least one perfluorinated moiety can in particular also be used.

The PCR pre-amplification is performed in a reaction volume that is preferably in the range of 10 μl to 50 μl, and particularly preferably in the range of 20 μl to 30 μl. More particularly preferably, it is 25 μl. Such a reaction volume is sufficient in order to supply a plurality of reaction compartments with pre-amplified sample portions and yet can be rapidly heated and cooled.

In order to perform a plurality of different singleplex detections, it is also preferred that the PCR pre-amplification be performed using 2 to 100 target-specific primer pairs, particularly preferably using 5 to 30 target-specific primer pairs. The PCR primers used in pre-amplification can also provide advantageous functionality in a subsequent isothermal detection. For example, the length of the primers can be advantageously designed to enable pre-amplification on the one hand and to additionally provide, for example, a “displacement” functionality in the isothermal detection reactions on the other hand.

In particular, the duplication of the nucleic acids in the course of pre-amplification takes place, taking into account the transfer efficiency achieved in microfluidic processing. In this way, a low transfer efficiency is already sufficient for the method to achieve, with sufficient pre-amplification, a sensitive detection of a plurality of gene targets.

In contrast to isothermal amplification systems of singleplex detections, PCR pre-amplification requires temperature cycles. In this case, the PCR pre-amplification preferably undergoes 5 to 30 temperature cycles and particularly preferably 12 to 20 temperature cycles. Temperature cycling preferably takes place by pumping the sample to be amplified back and forth between several, in particular two, chambers whose walls are kept at different, constant temperatures. Thus, even with a low thermal conductivity of the chamber walls, it is possible to change the temperature of the sample quickly since the chamber walls only have to be kept at a constant temperature and their temperature does not have to be changed cyclically.

Finally, it is preferred that between the PCR pre-amplification and the division of the sample, at least one constituent of the sample is inactivated. The inactivation relates in particular to at least one constituent of the sample that is disadvantageous for the isothermal detection. The inactivation can take place, for example, by diluting or tempering the sample, by changing the composition of its buffer solution, in particular by changing the pH value of the buffer solution, by enzymatically inactivating in particular uracil-containing PCR primers, by extracting reactants from the sample, or by extracting amplicons, i.e., duplicated nucleic acid sequences.

The microfluidic analysis device is configured to be operated by means of the method. Its substrate, which delimits the reaction chambers in which the method is performed, can in particular consist of a polymer, a glass, a metal or a semi-metal, such as silicon. The material may be porous and/or may have surface functionalization. Materials with poor thermal conductivity are also suitable for realizing the microfluidic analysis device since they do not significantly slow the operation of the microfluidic analysis device as a result of the isothermal amplification system of the singleplex detection.

### BRIEF DESCRIPTION OF THE DRAWINGS

Exemplary embodiments of the invention are illustrated in the drawings and are explained in more detail in the following description.

FIG. 1 shows a flow chart of an exemplary embodiment of the method according to the invention.

FIG. 2 shows a flow chart of another exemplary embodiment of the method according to the invention.

### EXEMPLARY EMBODIMENTS OF THE INVENTION

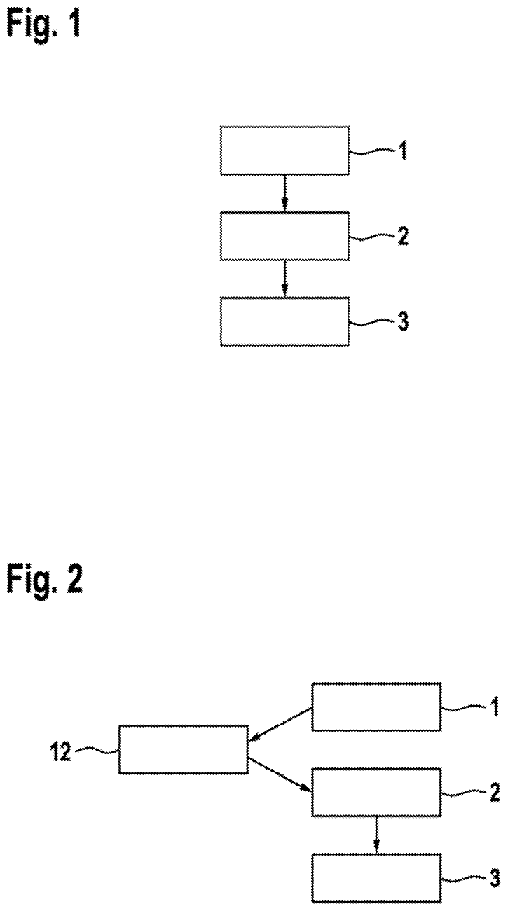

In an exemplary embodiment of the invention, a microfluidic analysis device is operated and is configured to perform multiplex PCR pre-amplification and several isothermal singleplex detections. A provided patient sample containing DNA strands to be examined is introduced into a first pre-amplification chamber having a volume of 25 μl. As shown in FIG. 1, a PCR pre-amplification of the sample is performed 1 first. For this purpose, the sample is mixed with 20 target-specific primer pairs, among other things, and subsequently undergoes 15 temperature cycles. For this purpose, it is pumped back and forth between the first pre-amplification chamber and a second pre-amplification chamber having the same volume. The first pre-amplification chamber is heated to a first temperature and the second pre-amplification chamber is brought to a second temperature. After completion of the PCR pre-amplification, the sample is divided 2 into 100 microfluidic reaction compartments each having a volume of 25 nl. The reaction compartments are designed as microcavities. Fluorinert FC-40 is used as transport and separating medium. The remainder of the sample, which is not divided into reaction compartments, is discarded or otherwise analyzed.

Finally, at least one singleplex detection is performed 3 in each of the reaction compartments. For example, SDA is used as an isothermal amplification system for this purpose. The detection of the different gene targets predetermined for the respective reaction compartments takes place, for example, in a fluorescence-based manner.

In a second exemplary embodiment of the method, which is shown in FIG. 2, after performing 1 the PCR pre-amplification and prior to dividing 2 the sample, constituents of the sample that would have a negative effect on the isothermal amplification by means of SDA are inactivated 12. To this end, the composition of the buffer solution is changed such that its pH value changes and enzymes are inactivated.

1. A method for operating a microfluidic analysis device, comprising:
providing a sample containing DNA;
performing a PCR pre-amplification of the sample;
dividing the sample into at least two reaction compartments; and
performing at least one singleplex detection in each of the at least two reaction compartments,
wherein the at least one singleplex detection is performed using an isothermal amplification system.

2. The method according to claim 1, wherein the isothermal amplification system is selected from a group consisting of SDA, RPA, LAMP, RCA, MDA, NASBA, HDA, NEAR, NESA, and HCR.

3. The method according to claim 1, wherein the at least two reaction compartments each have a volume of less than 1 μl.

4. The method according to claim 1, wherein the at least two reaction compartments comprise 2 to 500 reaction compartments into which the sample is divided.

5. The method according to claim 1, wherein the division of the sample takes place using at least partially fluorinated hydrocarbon.

6. The method according to claim 1, wherein the PCR pre-amplification is performed in a reaction volume in the range of 10 μl to 50 μl.

7. The method according to claim 1, wherein the PCR pre-amplification is performed using 2 to 100 target-specific primer pairs.

8. The method according to claim 1, wherein the PCR pre-amplification is performed cyclically, undergoing 5 to 30 temperature cycles, wherein the sample is pumped back and forth between several chambers.

9. The method according to claim 1, wherein between the PCR pre-amplification and the division of the sample, at least one constituent of the sample is inactivated.

10. A system comprising:
a microfluidic analysis device, configured to
receive a sample containing DNA,
perform a PCR pre-amplification of the sample,
divide the sample into at least two reaction compartments, and
perform at least one singleplex detection in each of the at least two reaction compartments,
wherein the at least one singleplex detection is performed using an isothermal amplification system.
